(19) United States
(12) Patent Application Publication
(10) Pub. No.: US 2021/0138445 A1
(43) Pub. Date: May 13, 2021

(54) A CATALYST FOR CONVERTING SYNTHESIS GAS TO ALCOHOLS

(71) Applicant: BASF SE, Ludwigshafen am Rhein (DE)

(72) Inventors: Christiane JANKE, Ludwigshafen am Rhein (DE); Ekkehard SCHWAB, Ludwigshafen am Rhein (DE); Stephan A. SCHUNK, Heidelberg (DE); Ivana JEVTOVIKJ, Heidelberg (DE); Harry KAISER, Heidelberg (DE); Frank ROSOWSKI, Ludwigshafen am Rhein (DE); Stefan ALTWASSER, Ludwigshafen am Rhein (DE); Virginie BETTE, Ludwigshafen am Rhein (DE)

(21) Appl. No.: 16/491,870

(22) PCT Filed: Mar. 9, 2018
(86) PCT No.: PCT/EP2018/055898

§ 371 Date: Sep. 6, 2019

(30) Foreign Application Priority Data
Mar. 10, 2017 (EP) 17160382.2

(51) Int. Cl. B01J 35/00; B01J 37/02; B01J 21/04; B01J 21/06; B01J 21/08; B01J 37/08; B01J 23/89; C07C 29/158

(52) U.S. Cl. CPC B01J 35/0006; B01J 37/0201; B01J 21/04; B01J 21/063; B01J 21/066; B01J 35/1019; B01J 37/088; B01J 23/8986; B01J 23/8953; C07C 29/158; B01J 21/08

(57) ABSTRACT

A catalyst for converting a synthesis gas, said catalyst comprising a first catalyst component and a second catalyst component, wherein the first catalyst component comprises, supported on a first porous oxidic substrate, Rh, Mn, an alkali metal M and Fe, and wherein the second catalyst component comprises, supported on a second porous oxidic support material, Cu and a transition metal other than Cu.

The present invention relates to a catalyst for converting a synthesis gas, said catalyst comprising a first catalyst component and a second catalyst component, wherein the first catalyst component comprises, supported on a first porous oxidic substrate, Rh, Mn, an alkali metal M and Fe, and wherein the second catalyst component comprises, supported on a second porous oxidic support material, Cu and a transition metal other than Cu. Further, the present invention relates to a process for preparing said catalyst and the use of said catalyst for converting a synthesis gas to one or more of methanol and ethanol. Yet further, the present invention relates to a reactor tube comprising said catalyst, and a reactor comprising said reactor tube.

The direct conversion of synthesis gas in one reactor to methanol and/or ethanol has a high technical potential as an alternative, low-cost route for producing said alcohols. Therefore, in order to achieve maximum economic benefits for said direct conversion of a synthesis gas to methanol and/or ethanol, high yields and selectivities regarding said alcohols have to be realized. On the other hand, not only the yields and selectivities regarding the alcohols have to be taken into account for an industrial-scale process, since it is also very important that the selectivities regarding by-products, in the present case in particular methane, should be kept as slow as possible.

Some catalysts for the direct conversion of synthesis gas in one reactor to methanol and/or ethanol are known in the art. Reference is made, for example, to US 2015/0284306 A1. Specifically, such catalysts typically contain Rh. Rh, however, is a very expensive metal, and in view of the maximum economic benefits mentioned above, the amount of Rh in a catalyst and a reactor bed, respectively, should be kept as low as possible.

Surprisingly, it was found that a catalyst having a specific composition and comprising two specific catalyst components solves one or more of these problems.

Therefore, the present invention relates to a catalyst for converting a synthesis gas, said catalyst comprising a first catalyst component and a second catalyst component, wherein the first catalyst component comprises, supported on a first porous oxidic substrate, Rh, Mn, an alkali metal M and Fe, and wherein the second catalyst component comprises, supported on a second porous oxidic support material, Cu and a transition metal other than Cu.

Preferably, in the first catalyst component, Rh, Mn, an alkali metal M and Fe are present as oxides. Prior to use, the catalyst of the present invention can be subjected to reduction in a reducing atmosphere, for example comprising hydrogen, wherein one or more of these oxides can be at least partially reduced to the respective metals. Such a reducing process preferably comprises bringing the catalyst in contact with a gas stream comprising hydrogen, wherein preferably at least 95 volume-%, preferably at least 98 volume-%, more preferably at least 99 weight-% of the gas stream consists of hydrogen. Preferably, the gas stream comprising hydrogen is brought in contact with the catalyst at a temperature of the gas stream in the range of from 250 to 350° C., more preferably in the range of from 275 to 325° C., preferably at a pressure of the gas stream in the range of from 10 to 100 bar(abs), more preferably in the range of from 20 to 80 bar(abs). Preferably, the catalyst is brought in contact with the gas stream comprising hydrogen for a period of time in the range of from 0.1 to 12 h, preferably in the range of from 0.5 to 6 h, more preferably in the range of from 1 to 3 h. Therefore, the present invention also relates to a catalyst which is obtainable or obtained or preparable or prepared by said reducing process.

In the first catalyst component, it is preferred that the molar ratio of Rh, calculated as elemental Rh, relative to Mn, calculated as elemental Mn, is in the range of from 0.1 to 10, preferably in the range of from 1 to 8, more preferably in the range of from 2 to 5. In the first catalyst component, it is preferred that the molar ratio of Rh, calculated as elemental Rh, relative to Fe, calculated as elemental Fe, is in the range of from 0.1 to 10, preferably in the range of from 1 to 8, more preferably in the range of from 2 to 5. In the first catalyst component, it is preferred that the molar ratio of Rh calculated as elemental Rh, relative to the alkali metal M, calculated as elemental M, is in the range of from 0.1 to 5, preferably in the range of from 0.15 to 3, more preferably in the range of from 0.25 to 2.5.

With regard to the alkali metal comprised in the first catalyst component, it is preferred that it is one or more of Na, Li, K, Rb, Cs, preferably one or more of Na, Li, and K. More preferably, the alkali metal M comprised in the first catalyst component comprises Li. More preferably, the alkali metal M comprised in the first catalyst component is Li. More preferably, the first catalyst component comprises any alkali metal, if present, only as unavoidable impurities, preferably in an amount of at most 100 weight-ppm, based on the total weight of the first catalyst component.

Therefore, it is preferred that the first catalyst component comprises Rh, Mn, Li and Fe, wherein

the molar ratio of Rh calculated as elemental Rh, relative to Fe, calculated as elemental Fe, is in the range of from 2 to 5,

the molar ratio of Rh calculated as elemental Rh, relative to Mn calculated as elemental Mn, is in the range of from 2 to 5, and

the molar ratio of Rh, calculated as elemental Rh, relative to Li, calculated as elemental Li, is in the range of from 0.25 to 2.5.

Generally, the first catalyst component may comprises one or more further components. Preferably, the first catalyst component essentially consists of the components mentioned above. Therefore, preferably at least 99 weight-%, more preferably at least 99.5 weight-%, more preferably at least 99.9 weight of the first catalyst component consist of Rh, Mn, the alkali metal M, Fe, O, and the first porous oxidic substrate.

If the first catalyst component comprises one or more further components, it is preferred that it comprises one or more further metals, more preferably one or more of Cu and Zn, wherein more preferably, the first catalyst component additionally comprises one further metal, more preferably Cu or Zn, wherein the one or more further metals are preferably present as oxides. If the first catalyst component comprises said further metal, it is preferred that the molar ratio of Rh, calculated as elemental Rh, relative to the further metal, calculated as elemental metal, preferably calculated as Cu and/or Zn, is in the range of from 0.1 to 5, preferably in the range of from 0.2 to 4, more preferably in the range of from 0.3 to 1.0. If the first catalyst component comprises the one or more further metals, it is preferred that the first catalyst component essentially consists of the components mentioned above and the one or more further metals. Therefore, in this case, it is preferred that at least 99 weight-%, more preferably at least 99.5 weight-%, more preferably at least 99.9 weight-% such as from 99.9 to 100 weight-% of the first catalyst component consist of Rh, Mn, the alkali metal M, Fe, O, the one or more further metals, preferably Cu or Zn, and the first porous oxidic substrate.

Regarding the first porous oxidic substrate, no specific restrictions exist, provided that the metals mentioned above can be supported on the substrate and that the resulting substrate can be used in the respectively desired application. Preferably, the first porous oxidic substrate comprises silica, zirconia, titania, alumina, a mixture of two or more of silica, zirconia, titania, and alumina, or a mixed oxide of two or more of silicon, zirconium, titanium, and aluminum, wherein more preferably, the first porous oxidic substrate comprises silica. More preferably, the first porous oxidic substrate essentially consists of silica. Therefore, preferably at least 99 weight-%, more preferably at least 99.5 weight-%, more preferably at least 99.9 weight-% such as from 99.9 to 100 weight-% of the first porous oxidic substrate consist of silica.

Generally, the amount of the metals supported on the first porous oxidic substrate are not subject to any specific restriction. Preferably, in the first catalyst component, the weight ratio of Rh, calculated as elemental Rh, relative to the first porous oxidic substrate is in the range of from 0.001:1 to 4.000:1, preferably in the range of from 0.005:1 to 0.200:1, more preferably in the range of from 0.010:1 to 0.070:1. The respective amounts of the other metals result from the respective weight ratios described above.

Preferably, the first catalyst component is essentially free of chlorine. Therefore, the chlorine content of first catalyst component, calculated as elemental CI, is in the range of from 0 to 100 weight-ppm based on the total weight of the first catalyst component.

Preferably, the first catalyst component is essentially free of titanium. Therefore, wherein the titanium content of first catalyst component, calculated as elemental Ti, is in the range of from 0 to 100 weight-ppm based on the total weight of the first catalyst component.

Preferably, the first catalyst component has a BET specific surface area in the range of from 250 to 500 m²/g, preferably in the range of from 300 to 475 m²/g, more preferably in the range of from 320 to 450 m²/g, determined as described in Reference Example 1.1 herein.

Preferably, the first catalyst component has a total intrusion volume in the range of from 0.1 to 5 mL/g, preferably in the range of from 0.5 to 3 mL/g, determined as described in Reference Example 1.2 herein.

Preferably, the first catalyst component has an average pore diameter in the range of from 0.001 to 0.5 micrometer, preferably in the range of from 0.01 to 0.05 micrometer, determined as described in Reference Example 1.3 herein.

With regard to the second catalyst component, the transition metal other than Cu preferably comprises one or more of Cr and Zn, more preferably is one or more of Cr and Zn. More preferably, in the second catalyst component, the transition metal other than Cu is Zn.

Preferably, in the second catalyst component, Cu and the transition metal other than Cu are present as oxides. Prior to use, the second catalyst component of the present invention can be subjected to reduction in a reducing atmosphere, for example comprising hydrogen, wherein one or more of these oxides can be at least partially reduced to the respective metals. Such a reducing process preferably comprises bringing the second catalyst component in contact with a gas stream comprising hydrogen, wherein preferably at least 95 volume-%, preferably at least 98 volume-%, more preferably at least 99 weight-% of the gas stream consists of hydrogen. Preferably, the gas stream comprising hydrogen is brought in contact with the second catalyst component at a temperature of the gas stream in the range of from 250 to 350° C., more preferably in the range of from 275 to 325° C., preferably at a pressure of the gas stream in the range of from 10 to 100 bar(abs), more preferably in the range of from 20 to 80 bar(abs). Preferably, the second catalyst component is brought in contact with the gas stream comprising hydrogen for a period of time in the range of from 0.1 to 12 h, preferably in the range of from 0.5 to 6 h, more preferably in the range of from 1 to 3 h. Therefore, the present invention also relates to a second catalyst component which is obtainable or obtained or preparable or prepared by said reducing process.

Preferably, in the second catalyst component, the molar ratio of Cu, calculated as elemental Cu, relative to the transition metal other than Cu, preferably Zn, calculated as elemental metal, preferably as Zn, is in the range of from 0.1 to 5, more preferably in the range of from 0.2 to 4, more preferably in the range of from 0.3 to 1.0.

Generally, the second catalyst component may comprise one or more further components. Preferably, the second catalyst component essentially consists of the components mentioned above. Therefore, preferably at least 99 weight-%, more preferably at least 99.5 weight-%, more preferably at least 99.9 weight-% such as from 99.9 to 100 weight-% of the second catalyst component consist of Cu, the transition metal other than Cu, 0, and the second porous oxidic substrate.

Regarding the second porous oxidic substrate, no specific restrictions exist, provided that the metals mentioned above can be supported on the substrate and that the resulting substrate can be used in the respectively desired application. Preferably, the second porous oxidic substrate comprises silica, zirconia, titania, alumina, a mixture of two or more of silica, zirconia, titania, and alumina, or a mixed oxide of two or more of silicon, zirconium, titanium, and aluminum, wherein more preferably, the second porous oxidic substrate comprises silica. More preferably, the second porous oxidic substrate essentially consists of silica. Therefore, preferably at least 99 weight-%, more preferably at least 99.5 weight-%, more preferably at least 99.9 weight-% such as from 99.9 to 100 weight-% of the second porous oxidic substrate consist of silica.

Generally, the amount of the metals supported on the second porous oxidic substrate is not subject to any specific restriction. Preferably, in the second catalyst component, the weight ratio of Cu, calculated as elemental Cu, relative to the second porous oxidic substrate, is in the range of from 0.001 to 0.5, preferably in the range of from 0.005 to 0.25, more preferably in the range of from 0.01 to 0.2. The respective amounts of the other metals or of the other metal result from the respective weight ratios described above.

Preferably, the second catalyst component has a BET specific surface area in the range of from 100 to 500 m²/g, more preferably in the range of from 159 to 425 m²/g, more preferably in the range of from 200 to 350 m²/g, determined as described in Reference Example 1.1 herein.

Preferably, the second catalyst component has a total intrusion volume in the range of from 0.1 to 10 mL/g, preferably in the range of from 0.5 to 5 mL/g, determined as described in Reference Example 1.2 herein.

Preferably, the second catalyst component has an average pore diameter in the range of from 0.001 to 5 micrometer, preferably in the range of from 0.01 to 2.5 micrometer, determined as described in Reference Example 1.3 herein.

With regard to the weight ratio of the first catalyst component relative to the second catalyst component in the catalyst of the present invention, no specific restrictions exist. Generally, the weight ratio can be adjusted to the respective needs. Preferably, the weight ratio of the first catalyst component relative to the second catalyst component is in the range of from 1 to 10, preferably in the range of from 1.5 to 8; more preferably in the range of from 2 to 6.

Generally, the catalyst of the present invention may comprise one or more further components in addition to the first catalyst component and the second catalyst component. Preferably, the catalyst essentially consists of the first catalyst component and the second catalyst component. Therefore, preferably at least 99 weight-%, more preferably at least 99.5 weight-%, more preferably at least 99.9 weight-% such as from 99.9 to 100 weight-% of the catalyst consist of the first catalyst component and the second catalyst component.

The present invention further relates to a reactor tube for converting a synthesis gas, comprising a catalyst bed which comprises the catalyst as described above. Generally, it is conceivable that the reactor tube comprising the catalyst bed is arranged horizontally so that a gas stream comprising a synthesis gas is passed through the reactor tube and, thus, through the catalyst bed, in horizontal direction. Preferably, the reactor tube comprising the catalyst bed is arranged vertically. Therefore, it is preferred that a gas stream comprising a synthesis gas is passed through the reactor tube and, thus, through the catalyst bed, in vertical direction, such as from the bottom of the reactor tube to the top thereof or from top of the reactor tube to the bottom thereof. With regard to the geometry of the reactor tube, no specific restrictions exist. Regarding, for example, the length of the reactor tube and the length of the catalyst bed comprised in the reactor tube, can be adjusted to the respective needs. Regarding, for example, the cross section of the reactor tube and the cross section of the catalyst bed, it may be preferred that it is of circular shape. Further, it is possible that the reaction tube is equipped with means suitable for heating and/or cooling the reaction tube, for example external means such as one or more jackets through which one or more cooling or heating media can be passed. Such heating and/or cooling means may be used, for example, to achieve an essentially isothermal reaction in the catalyst bed, i.e. to allow for isothermally converting the synthesis gas in the reactor tube.

Preferably, the catalyst bed comprised in the tube comprises two or more catalyst bed zones, such as two, three, or four catalyst bed zones, preferably two or three catalyst bed zones, more preferably two catalyst bed zones, wherein between two adjacent catalyst bed zones, it may be conceivable that an inert zone is arranged which may comprise, for example, alumina such as alpha alumina. More preferably, two adjacent catalyst bed zones are directly adjacent to each other, and specifically, no inert zone is arranged between said two zones. Such adjacent catalyst bed zones are realized in that a first catalyst is filled into the tube, and thereafter, a second catalyst is filled on top of the first catalyst, resulting in a reactor tube comprising two or more catalyst bed zones, wherein a first catalyst bed zone is arranged on top of a second catalyst bed zone, in particular if the reactor tube is arranged vertically. Preferably, the catalyst bed consists of the first catalyst bed zone and the second catalyst bed zone.

According to a first preferred embodiment, the first catalyst bed zone may comprise a first or a second catalyst component as described wherein it is preferred that the first catalyst bed zone comprises a second catalyst component as described above. More preferably, the first catalyst bed zone consists of a second catalyst component a described above. Preferably, the second catalyst bed zone comprises the catalyst comprising a first catalyst component and a second catalyst component as described above. More preferably, the second catalyst bed zone consists of the catalyst comprising a first catalyst component and a second catalyst component as described above. Generally, the second catalyst component of the catalyst and the second catalyst component of the first catalyst bed zone may have the same or a different composition. Preferably, the second catalyst component of the catalyst and the second catalyst component of the first catalyst bed zone have the same composition.

Generally, the amount of the catalyst in the second catalyst bed zone and the amount of the second catalyst component in the first catalyst bed zone may be chosen according to the specific needs. Preferably, the volume of the first catalyst bed zone relative to the volume of the second catalyst bed zone is in the range of from 0 to 100, more preferably in the range of from 0.01 to 50, more preferably in the range of from 0.5 to 5.

Therefore, the present invention preferably relates to a vertically arranged reactor tube comprising a catalyst bed consisting of a first catalyst bed zone arranged on top of a second catalyst bed zone, wherein the first catalyst bed zone consists of a second catalyst component as described above and wherein the second catalyst bed zone consists of a catalyst comprising a first catalyst component and a second catalyst component as described above, wherein the volume of the first catalyst bed zone relative to the volume of the second catalyst bed zone is in the range of from 0.5:1 to 5:1.

According to a second embodiment, the second catalyst bed zone may comprise a first or a second catalyst component as described wherein it is preferred that the second catalyst bed zone comprises a second catalyst component as described above. More preferably, the second catalyst bed zone consists of a second catalyst component a described above. Preferably, the first catalyst bed zone comprises the catalyst comprising a first catalyst component and a second catalyst component as described above. More preferably, the first catalyst bed zone consists of the catalyst comprising a first catalyst component and a second catalyst component as described above. Generally, the second catalyst component of the catalyst and the second catalyst component of the first catalyst bed zone may have the same or a different composition. Preferably, the second catalyst component of the catalyst and the second catalyst component of the first catalyst bed zone have the same composition.

Generally, the amount of the catalyst in the first catalyst bed zone and the amount of the second catalyst component in the second catalyst bed zone may be chosen according to the specific needs. Preferably, the volume of the first catalyst bed zone relative to the volume of the second catalyst bed zone is in the range of from 0 to 100, more preferably in the range of from 0.01 to 50, more preferably in the range of from 0.5 to 5.

Further, the present invention relates to a catalyst bed comprising a first catalyst bed zone and a second catalyst bed zone described above.

Preferably, the reactor tube described above has inlet means allowing a gas stream to be passed into the reactor tube and outlet means allowing a gas stream to be removed from the reactor tube. More preferably, the vertically arranged reactor tube has inlet means at the top allowing a gas stream to be passed into the reactor tube and outlet means at the bottom allowing a gas stream to be removed from the reactor tube.

The present invention further relates to a reactor for converting a synthesis gas, comprising one or more reactor tubes as described above wherein the one or more reactor tubes are preferably vertically arranged. Preferably, the vertically arranged reactor tubes have inlet means at the top allowing a gas stream to be passed into the reactor tube and outlet means at the bottom allowing a gas stream to be removed from the reactor tube. The reactor may comprise two or more reactor tubes as described above, wherein the two or more reactor tubes are preferably arranged in parallel. Further, the reactor may comprise temperature adjustment means allowing for isothermally converting the synthesis gas in the one or more reactor tubes.

The present invention further relates to the use of the catalyst as described above, optionally in combination with a second catalyst component according to any one of embodiments 1 and 18 to 27, for converting a synthesis gas comprising hydrogen and carbon monoxide, preferably for converting synthesis gas comprising hydrogen and carbon monoxide to one or more alcohols, preferably one or more of methanol and ethanol. According to said use, it is preferred that for converting, the synthesis gas is passed into a reactor tube as described above, wherein said reactor tube may be comprised in a reactor as described above. Further according to said use, it is preferred that the synthesis gas is passed into the reactor tube together with an inert gas, said inert gas preferably comprising argon.

The present invention further relates to a process for converting a synthesis gas comprising hydrogen and carbon monoxide to one or more of methanol and ethanol, said process comprising
(i) providing a gas stream which comprises a synthesis gas stream comprising hydrogen and carbon monoxide;
(ii) providing a catalyst as described above and optionally a second catalyst component as described above;
(iii) bringing the gas stream provided in (i) in contact with the catalyst provided in (ii) and optionally the second catalyst component, obtaining a reaction mixture stream comprising one or more of methanol and ethanol.

Generally, the process can be carried out in any suitable manner. Preferably, the catalyst provided in (ii) is comprised in a reactor tube as described above, wherein said reactor tube is preferably comprised in a reactor as descried above. More preferably, bringing the gas stream provided in (i) in contact with the catalyst provided in (ii) according to (iii) comprises passing the gas stream as feed stream into the reactor tube and through the catalyst bed comprised in the reactor tube, preferably from the top of the reactor tube to the bottom of the reactor tube, obtaining the reaction mixture stream comprising one or more of methanol and ethanol. Further, said process preferably comprises
(iv) removing the reaction mixture stream obtained from (iii) from the reactor tube.

With regard to the composition of the synthesis gas, no specific restrictions exist. Preferably, in the synthesis gas stream provided in (i), the molar ratio of hydrogen relative to carbon monoxide is in the range of from 0.5:1 to 10:1, more preferably in the range of from 1:1 to 8:1, more preferably in the range of from 1.5:1 to 6:1, more preferably in the range of from 2:1 to 5:1.

According to a first preferred embodiment, in the synthesis gas stream provided in (i), the molar ratio of hydrogen relative to carbon monoxide is in the range of from 1:1 to 3:1, preferably in the range of from 1.5:1 to 2.5:1, more preferably in the range of from 1.75:1 to 2.25:1. According to a second preferred embodiment, in the synthesis gas stream provided in (i), the molar ratio of hydrogen relative to carbon monoxide is in the range of from 4:1 to 6:1, preferably in the range of from 4.5:1 to 5.5:1, more preferably in the range of from 4.75:1 to 5.25:1.

Generally, the synthesis gas stream may comprise one or more further components in addition to hydrogen and carbon monoxide. Preferably, the synthesis gas stream essentially consists of hydrogen and carbon monoxide. Therefore, preferably at least 99 volume-%, more preferably at least 99.5 volume-%, more preferably at least 99.9 volume-% of the synthesis gas stream according to (i) consist of hydrogen and carbon monoxide.

Generally, the gas stream may provided in (i) comprise one or more further components in addition to synthesis gas stream. According to a first preferred embodiment, the gas stream essentially consists of the synthesis gas stream. Therefore, preferably at least 80 volume-%, preferably at least 85 volume-%, more preferably at least 90 volume-% such as from 90 to 99 volume-% of the gas stream provided in (i) consist of the synthesis gas stream. Further, it is possible that at least 99 volume-%, preferably at least 99.5 volume-%, more preferably at least 99.9 volume-% such as from 99.9 to 100 volume-% of the gas stream provided in (i) consist of the synthesis gas stream.

According to second preferred embodiment, the gas stream provided in (i) further comprises one or more inert gases. No specific restrictions exist with regard to the chemical nature of the one or more further inert gases provided they are inert or essentially inert in the reaction according to (iii). Preferably, the one or more inert gases comprises argon. More preferably, the one or more inert gases is argon. According to the second preferred embodiment, it is preferred that in the gas stream provided in (i), the volume ratio of the one or more inter gases relative to the synthesis gas stream is in the range of from 1:20 to 1:2, preferably in the range of from 1:15 to 1:5, more preferably in the range of from 1:12 to 1:8. Further according to the second preferred embodiment, it is preferred that at least 99 volume-%, more preferably at least 99.5 volume-%, more preferably at least 99.9 volume-% of the gas stream provided in (i) consist of the synthesis gas stream and the one or more inert gases.

Bringing the gas stream in contact with the catalyst according to (iii) is preferably carried out at a temperature of the gas stream in the range of from 200 to 400° C., more preferably in the range of from 220 to 350° C., more preferably in the range of from 240 to 310° C. Conceivable preferred ranges are from 240 to 290° C. or from 240 to 270° C. Further, bringing the gas stream in contact with the catalyst according to (iii) is preferably carried out at a pressure of the gas stream in the range of from 20 to 100 bar(abs), more preferably in the range of from 40 to 80 bar(abs), more preferably in the range of from 50 to 60 bar(abs). Yet further, bringing the gas stream in contact with the catalyst according to (iii) is preferably carried out at a gas hourly space velocity in the range of from 100 to 25,000 h⁻¹, preferably in the range of from 500 to 20,000 h⁻¹, more preferably in the range of from 1,000 to 10,000 h⁻¹, wherein the gas hourly space velocity is defined as the volume flow rate of the gas stream brought in contact with the catalyst divided by the volume of the catalyst bed.

According to the present invention, it is preferred that the catalyst, provided in (i), is suitably reduced prior to (iii), the catalyst provided in (i) is reduced. Generally, reducing the catalyst can be carried out in any suitable vessel wherein it is preferred that the catalyst is reduced in the reactor tube in which the reaction according to (iii) is carried out. If a first or a second catalyst component, preferably a second catalyst component is present in the catalyst bed in addition to the catalyst, preferably in a separate catalyst bed zone as described above, it is preferred that also said first or second catalyst component is reduced prior to (iii), more preferably at the same conditions at which the catalyst is reduced. Regarding the reducing conditions, no specific restrictions exist. Preferably, reducing the catalyst comprises bringing the catalyst in contact with a gas stream comprising hydrogen, wherein preferably at least 95 volume-%, more preferably at least 98 volume-%, more preferably at least 99 weight-% of the gas stream consists of hydrogen. Preferably, said gas stream comprising hydrogen is brought in contact with the catalyst at a temperature of the gas stream in the range of from 250 to 350° C., more preferably in the range of from 275 to 325° C. Preferably, said gas stream comprising hydrogen is brought in contact with the catalyst at a pressure of the gas stream in the range of from 10 to 100 bar(abs), preferably in the range of from 20 to 80 bar(abs). Preferably, the gas stream comprising hydrogen is brought in contact with the catalyst at a gas hourly space velocity in the range of from 500 to 15,000 h⁻¹, preferably in the range of from 1,000 to 10,000 h⁻¹, more preferably in the range of from 2,000 to 8,000 h⁻¹, wherein the gas hourly space velocity is defined as the volume flow rate of the gas stream brought in contact with the catalyst divided by the volume of the catalyst bed. Preferably, the catalyst is brought in contact with the gas stream comprising hydrogen for a period of time in the range of from 0.1 to 12 h, preferably in the range of from 0.5 to 6 h, more preferably in the range of from 1 to 3 h.

The process of the present invention is characterized by a high selectivity towards the one or more of methanol and ethanol, and simultaneously by a low selectivity towards towards undesired by-products such as methane and acetic acid, in particular methane, wherein these selectivities are observed in a wide temperature range of the reaction.

In particular, the conversion of the synthesis gas to one or more of methanol and ethanol preferably preferably exhibits a selectivity towards methane of at most 15% at a temperature during conversion of 260° C., preferably exhibits a selectivity towards methane of at most 25% at a temperature during conversion of 280° C., and preferably exhibits a selectivity towards methane of at most 35% at a temperature during conversion of 300° C. With regard to the by-product acetic acid, the conversion of the synthesis gas to one or more of methanol and ethanol preferably exhibits a selectivity towards acetic acid of less than 1% at a temperature during conversion of 260° C. or 280° C. or 300° C. Yet further, the conversion of the synthesis gas to one or more of methanol and ethanol preferably exhibits a selectivity towards the one or more of methanol and ethanol of at least 50% at a temperature during conversion of 260° C., and preferably exhibits a selectivity towards the one or more of methanol and ethanol of at least 45% at a temperature during conversion of 280° C.

Generally, the catalyst of the present invention can be prepared by any suitable process. Preferably, said process comprises
(a) providing the first catalyst component as described above;
(b) providing the second catalyst component as described above;
(c) mixing the first catalyst component provided in (a) and the second catalyst component provided in (b).

Preferably, providing the first catalyst component according to (a) comprises preparing the first catalyst component by a method comprising
(a.1) providing a source of the first porous oxidic substrate, preferably comprising subjecting the source of the first porous oxidic substrate to calcination;
(a.2) providing a source of Rh, a source of Mn, a source of the alkali metal, preferably Li, and a source of Fe;
(a.3) impregnating the preferably calcined source of the first porous oxidic substrate obtained from (a.1) with the sources provided in (a.2);
(a.4) calcining the impregnated source of the first porous oxidic substrate, preferably after drying.

Preferably, according to (a.1), the first porous oxidic substrate is calcined in a gas atmosphere at a temperature of the gas atmosphere in the range of from 450 to 650° C., preferably in the range of from 500 to 600° C., wherein the gas atmosphere preferably comprises oxygen, more preferably is oxygen, air, or lean air. The source of the first porous oxidic substrate according to (a.1) preferably comprises silica, zirconia, titania, alumina, a mixture of two or more of silica, zirconia, titania, and alumina, or a mixed oxide of two or more of silicon, zirconium, titanium, and aluminum. More preferably, the first porous oxidic substrate comprises silica. More preferably, at least 95 weight-%, more preferably at least 98 weight-%, more preferably at least 99 weight-% of the first porous oxidic substrate consist of silica. Preferably, the silica, preferably subjected to calcination as described above, has a BET specific surface area in the range of 500 to 550 m²/g. Further, the silica preferably has a total intrusion volume in the range of from 0.70 to 0.80 mL/g. Yet further, the silica preferably has an average pore diameter in the range of from 55 to 65 Angstrom.

Regarding the sources of the metals, no specific restrictions exist. Preferably, the source of Rh comprises a Rh salt, more preferably an inorganic Rh salt, more preferably a Rh nitrate, wherein more preferably, the source of Rh is a Rh nitrate. Preferably, the source of Mn comprises a Mn salt, more preferably an inorganic Mn salt, more preferably a Mn nitrate, wherein more preferably, the source of Mn is a Rh nitrate. Preferably, the source of the alkali metal, preferably Li, comprises an alkali metal salt, preferably a Li salt, more preferably an inorganic alkali metal salt, preferably an inorganic Li salt, more preferably an alkali metal nitrate, preferably a Li nitrate, wherein more preferably, the source of the alkali metal is an alkali metal nitrate, more preferably a Li nitrate. Preferably, the source of Fe comprises a Fe salt, more preferably an inorganic Fe salt, more preferably a Fe nitrate, wherein more preferably, the source of Fe is a Fe nitrate.

Providing the sources according to (a.2) preferably comprises preparing an aqueous solution comprising the source of Rh, the source of Mn, the source of the alkali metal, preferably Li, and the source of Fe. The respective amounts of the sources are suitably chosen by the skilled person so that the desired preferred amounts of the metals, as described above, are obtained by the preparation process. Preferably, according to (a.3), the source of the first porous oxidic substrate obtained from (a.1) is impregnated with said aqueous solution.

According to (a.4), it is preferred that the impregnated source of the first porous oxidic substrate obtained from (a.3) is calcined in a gas atmosphere at a temperature of the gas atmosphere in the range of from 180 to 250° C., more preferably in the range of from 190 to 220° C., wherein the gas atmosphere preferably comprises oxygen, more preferably is oxygen, air, or lean air. Preferably, prior to calcining, the impregnated source of the first porous oxidic substrate obtained from (a.3) is dried in a gas atmosphere at a temperature of the gas atmosphere in the range of from 90 to 150° C., preferably in the range of from 100 to 130° C., wherein the gas atmosphere preferably comprises oxygen, more preferably is oxygen, air, or lean air.

Preferably, providing the second catalyst component according to (b) comprises preparing the second catalyst component by a method comprising
(b.1) providing a source of the second porous oxidic substrate, preferably comprising subjecting the source of the second porous oxidic substrate to calcination;
(b.2) preparing a source of Cu, a source of the transition metal other than Cu, preferably Zn;
(b.3) impregnating the preferably calcined source of the second porous oxidic substrate obtained from (a.1) with the sources preparing in (a.2);
(b.4) calcining the impregnated source of the second porous oxidic substrate, preferably after drying.

Preferably, according to (b.1), the second porous oxidic substrate is calcined in a gas atmosphere at a temperature of the gas atmosphere in the range of from 750 to 950° C., preferably in the range of from 800 to 900° C., wherein the gas atmosphere preferably comprises oxygen, more preferably is oxygen, air, or lean air. The source of the second porous oxidic substrate according to (b.1) preferably comprises silica, zirconia, titania, alumina, a mixture of two or more of silica, zirconia, titania, and alumina, or a mixed oxide of two or more of silicon, zirconium, titanium, and aluminum. More preferably, the second porous oxidic substrate comprises silica. More preferably, at least 95 weight-%, more preferably at least 98 weight-%, more preferably at least 99 weight-% of the second porous oxidic substrate consist of silica. Preferably, the silica, preferably subjected to calcination as described above, has a BET specific surface area in the range of 500 to 550 m²/g. Further, the silica preferably has a total intrusion volume in the range of from 0.70 to 0.80 mL/g. Yet further, the silica preferably has an average pore diameter in the range of from 55 to 65 Angstrom.

Regarding the sources of the transition metals, no specific restrictions exist. Preferably, the source of Cu comprises a Cu salt, more preferably an inorganic Cu salt, more preferably a Cu nitrate, wherein more preferably, the source of Cu is a Cu nitrate. Preferably, the source of the transition metal other than Cu, preferably Zn, comprises a salt of the transition metal other than Cu, preferably a Zn salt, more preferably an inorganic salt of the transition metal other than Cu, preferably an inorganic Zn salt, more preferably a nitrate of the transition metal other than Cu, preferably a Zn nitrate, wherein more preferably, the source of the transition metal other than Cu is a nitrate of the transition metal other than Cu, more preferably a Zn nitrate.

Providing the sources according to (b.2) preferably comprises preparing an aqueous solution comprising the source of Cu and the source of the transition metal other than Cu, preferably Zn. The respective amounts of the sources are suitably chosen by the skilled person so that the desired preferred amounts of the transition metals, as described above, are obtained by the preparation process. Preferably, according to (b.3), the source of the second porous oxidic substrate obtained from (b.1) is impregnated with said aqueous solution.

According to (b.4), it is preferred that the impregnated source of the second porous oxidic substrate obtained from (b.3) is calcined in a gas atmosphere at a temperature of the gas atmosphere in the range of from 300 to 500° C., more preferably in the range of from 350 to 450° C., wherein the gas atmosphere preferably comprises oxygen, more preferably is oxygen, air, or lean air. Preferably, prior to calcining, the impregnated source of the second porous oxidic substrate obtained from (b.3) is dried in a gas atmosphere at a temperature of the gas atmosphere in the range of from 80 to 140° C., preferably in the range of from 90 to 120° C., wherein the gas atmosphere preferably comprises oxygen, more preferably is oxygen, air, or lean air.

The present invention further relates to the first catalyst component as described above, which is obtainable or obtained or preparable or prepared by a process as described above, said process preferably comprising (a.1), (a.2), (a.3) and (a.4). The present invention yet further relates to the second catalyst component as described above, which is obtainable or obtained or preparable or prepared by a process as described above, said process preferably comprising (b.1), (b.2), (b.3) and (b.4).

Still further, the present invention relates to a porous oxidic substrate, comprising supported thereon Rh, Mn, Li and Fe, having a chlorine content, calculated as elemental CI, in the range of from 0 to 100 weight-ppm, based on the total weight of said substrate, Rh, Mn, Li and Fe, wherein said porous oxidic substrate is preferably obtainable or obtained or preparable or prepared by a process as described above, comprising (a.1), (a.2), (a.3) and (a.4). Preferably, said porous oxidic substrate is silica comprising supported thereon Rh, Mn, Li and Fe. More preferably, said porous oxidic substrate has a Rh content, calculated as elemental Rh, in the range of from 2.0 to 3.0 weight-%, more preferably in the range of from 2.1 to 2.8 weight-%, more preferably in the range of from 2.2 to 2.6 weight-%; a Mn content, calculated as elemental Mn, in the range of from 0.40 to 0.70 weight-%, more preferably in the range of from 0.45 to 0.60 weight-%, more preferably in the range of from 0.50 to 0.55 weight-%; a Fe content, calculated as elemental Li, in the range of from 0.35 to 0.65 weight-%, more preferably in the range of from 0.40 to 0.55 weight-%, more preferably in the range of from 0.45 to 0.50 weight-%; a Li content, calculated as elemental Li, in the range of from 0.10 to 0.40 weight-%, preferably in the range of from 0.15 to 0.30 weight-%, more preferably in the range of from 0.20 to 0.25 weight-%; in each case based on the total weight of the porous oxidic substrate, comprising supported thereon Rh, Mn, Li and Fe. Preferably at least 99 weight-%, more preferably at least 99.9 weight-%, more preferably at least 99.99 weight-% of the porous oxidic substrate consist of the porous oxidic substrate, Rh, Mn, Li and Fe. Said porous oxidic substrate preferably has a BET specific surface area in the range of from 350 to 450 m²/g, more preferably in the range of from 375 to 425 m²/g.

The present invention is further illustrated by the following embodiments and combinations of embodiments as indicated by the respective dependencies and back-references. In particular, it is noted that in each instance where reference is made to more than two embodiments, for example in the context of a term such as “The catalyst of any one of embodiments 1 to 4”, every embodiment in this range is meant to be explicitly disclosed, i.e. the wording of this term is to be understood as being synonymous to “The catalyst of any one of embodiments 1, 2, 3, and 4”.
1. A catalyst for converting a synthesis gas, said catalyst comprising a first catalyst component and a second catalyst component, wherein the first catalyst component comprises, supported on a first porous oxidic substrate, Rh, Mn, an alkali metal M and Fe, and wherein the second catalyst component comprises, supported on a second porous oxidic support material, Cu and a transition metal other than Cu.
2. The catalyst of embodiment 1, wherein in the first catalyst component, Rh, Mn, an alkali metal M and Fe are present as oxides.
3. The catalyst of embodiment 1 or 2, wherein in the first catalyst component,
   the molar ratio of Rh, calculated as elemental Rh, relative to Mn, calculated as elemental Mn, is in the range of from 0.1 to 10, preferably in the range of from 1 to 8, more preferably in the range of from 2 to 5;
   the molar ratio of Rh, calculated as elemental Rh, relative to Fe, calculated as elemental Fe, is in the range of from 0.1 to 10, preferably in the range of from 1 to 8, more preferably in the range of from 2 to 5, and
   the molar ratio of Rh calculated as elemental Rh, relative to the alkali metal M, calculated as elemental M, is in the range of from 0.1 to 5, preferably in the range of from 0.15 to 3, more preferably in the range of from 0.25 to 2.5.
4. The catalyst of any one of embodiments 1 to 3, wherein the alkali metal M comprised in the first catalyst component is one or more of Na, Li, K, Rb, Cs, preferably one or more of Na, Li, and K, wherein more preferably, the alkali metal M comprised in the first catalyst component comprises, more preferably is Li.
5. The catalyst of any one of embodiments 1 to 4, wherein the first catalyst component comprises Rh, Mn, Li and Fe, wherein
   the molar ratio of Rh calculated as elemental Rh, relative to Fe, calculated as elemental Fe, is in the range of from 2 to 5,
   the molar ratio of Rh calculated as elemental Rh, relative to Mn calculated as elemental Mn, is in the range of from 2 to 5, and
   the molar ratio of Rh, calculated as elemental Rh, relative to Li, calculated as elemental Li, is in the range of from 0.25 to 2.5.
6. The catalyst of any one of embodiments 1 to 5, wherein at least 99 weight-%, preferably at least 99.5 weight-%, more preferably at least 99.9 weight of the first catalyst component consist of Rh, Mn, the alkali metal M, Fe, O, and the first porous oxidic substrate.
7. The catalyst of any one of embodiments 1 to 6, wherein the first catalyst component additionally comprises one or more further metals, preferably one or more of Cu and Zn, wherein more preferably, the first catalyst component additionally comprises one further metal, more preferably Cu or Zn, wherein the one or more further metals are preferably present as oxides.
8. The catalyst of embodiment 7, wherein in the first catalyst component, the molar ratio of Rh, calculated as elemental Rh, relative to the further metal, calculated as elemental metal, preferably calculated as Cu and/or Zn, is in the range of from 0.1 to 5, preferably in the range of from 0.2 to 4, more preferably in the range of from 0.3 to 1.0.
9. The catalyst of embodiment 7 or 8, wherein at least 99 weight-%, preferably at least 99.5 weight-%, more preferably at least 99.9 weight-% of the first catalyst component consist of Rh, Mn, the alkali metal M, Fe, O, the one or more further metals, preferably Cu or Zn, and the first porous oxidic substrate.
10. The catalyst of any one of embodiments 1 to 9, wherein the first porous oxidic substrate comprises silica, zirconia, titania, alumina, a mixture of two or more of silica, zirconia, titania, and alumina, or a mixed oxide of two or more of silicon, zirconium, titanium, and aluminum, wherein more preferably, the first porous oxidic substrate comprises silica.
11. The catalyst of any one of embodiments 1 to 10, wherein at least 99 weight-%, preferably at least 99.5 weight-%, more preferably at least 99.9 weight-% of the first porous oxidic substrate consist of silica.
12. The catalyst of any one of embodiments 1 to 11, wherein in the first catalyst component, the weight ratio of Rh, calculated as elemental Rh, relative to the first porous oxidic substrate is in the range of from 0.001:1 to 4.000:1, preferably in the range of from 0.005:1 to 0.200:1, more preferably in the range of from 0.010:1 to 0.070:1.
13. The catalyst of any one of embodiments 1 to 12, wherein the chlorine content of first catalyst component is in the range of from 0 to 100 weight-ppm based on the total weight of the first catalyst component.
14. The catalyst of any one of embodiments 1 to 13, wherein the titanium content of first catalyst component is in the range of from 0 to 100 weight-ppm based on the total weight of the first catalyst component.
15. The catalyst of any one of embodiments 1 to 14, wherein the first catalyst component has a BET specific surface area in the range of from 250 to 500 m²/g, preferably in the range of from 320 to 450 m²/g, determined as described in Reference Example 1.1 herein.
16. The catalyst of any one of embodiments 1 to 15, wherein the first catalyst component has a total intrusion volume in the range of from 0.1 to 5 mL/g, preferably in the range of from 0.5 to 3 mL/g, determined as described in Reference Example 1.2 herein.
17. The catalyst of any one of embodiments 1 to 16, wherein the first catalyst component has an average pore diameter in the range of from 0.001 to 0.5 micrometer, preferably in the range of from 0.01 to 0.05 micrometer, determined as described in Reference Example 1.3 herein.
18. The catalyst of any one of embodiments 1 to 17, wherein in the second catalyst component, the transition metal other than Cu is one or more of Cr and Zn.
19. The catalyst of any one of embodiments 1 to 18, wherein in the second catalyst component, the transition metal other than Cu is Zn.
20. The catalyst of any one of embodiments 1 to 19, wherein in the second catalyst component, Cu and the transition metal other than Cu are present as oxides.
21. The catalyst of any one of embodiments 1 to 20, wherein in the second catalyst component, the molar ratio of Cu, calculated as elemental Cu, relative to the transition metal other than Cu, preferably Zn, calculated as elemental metal, preferably as Zn, is in the range of from 0.1 to 5, more preferably in the range of from 0.2 to 4, more preferably in the range of from 0.3 to 1.0.
22. The catalyst of any one of embodiments 1 to 21, wherein at least 99 weight-%, preferably at least 99.5 weight-%, more preferably at least 99.9 weight-% of the second catalyst component consist of Cu, the transition metal other than Cu, 0, and the second porous oxidic substrate.
23. The catalyst of any one of embodiments 1 to 22, wherein the second porous oxidic substrate comprises silica, zirconia, titania, alumina, a mixture of two or more of silica, zirconia, titania, and alumina, or a mixed oxide of two or more of silicon, zirconium, titanium, and aluminum, wherein more preferably, the second porous oxidic substrate comprises silica.
24. The catalyst of any one of embodiments 1 to 23, wherein at least 99 weight-%, preferably at least 99.5 weight-%, more preferably at least 99.9 weight-% of the second porous oxidic substrate consist of silica.
25. The catalyst of any one of embodiments 1 to 24, wherein in the second catalyst component, the weight ratio of Cu, calculated as elemental Cu, relative to the second porous oxidic substrate is in the range of from 0.001 to 0.5, preferably in the range of from 0.005 to 0.25, more preferably in the range of from 0.01 to 0.20.
26. The catalyst of any one of embodiments 1 to 25, wherein the second catalyst component has a BET specific surface area in the range of from 100 to 500 m²/g, preferably in the range of from 200 to 350 m²/g, determined as described in Reference Example 1.1 herein.
27. The catalyst of any one of embodiments 1 to 26, wherein the second catalyst component has a total intrusion volume in the range of from 0.1 to 10 mL/g, preferably in the range of from 0.5 to 5 mL/g, determined as described in Reference Example 1.2 herein; and wherein the second catalyst component has an average pore diameter in the range of from 0.001 to 5 micrometer, preferably in the range of from 0.01 to 2.5 micrometer, determined as described in Reference Example 1.3 herein.
28. The catalyst of any one of embodiments 1 to 27, wherein the weight ratio of the first catalyst component relative to the second catalyst component is in the range of from 1 to 10, preferably in the range of from 1.5 to 8; more preferably in the range of from 2 to 6.
29. The catalyst of any one of embodiments 1 to 28, wherein at least 99 weight-%, preferably at least 99.5 weight-%, more preferably at least 99.9 weight-% of the catalyst consist of the first catalyst component and the second catalyst component.
30. A reactor tube for converting a synthesis gas, comprising a catalyst bed which comprises the catalyst of any one of embodiments 1 to 29.
31. The reactor tube of embodiment 30, being vertically arranged.
32. The reactor tube of embodiment 30 or 31, having a circular cross section.
33. The rector tube of any one of embodiments 30 to 32, comprising two or more catalyst bed zones, wherein a first catalyst bed zone is arranged on top of a second catalyst bed zone.
34. The reactor tube of embodiment 33, wherein the first catalyst bed zone comprises, preferably consists of a second catalyst component according to any one of embodiments 1 and 18 to 27.
35. The reactor tube of embodiment 34, wherein the second catalyst bed zone comprises, preferably consists of the catalyst according to any one of embodiments 1 to 29.
36. The reactor tube of embodiment 34 or 35, wherein the volume of the first catalyst bed zone relative to the volume of the second catalyst bed zone is in the range of from 0 to 100, preferably in the range of from 0.01 to 50, more preferably in the range of from 0.5 to 5.
37. The rector tube of embodiment 33, wherein the first catalyst bed zone comprises, preferably consists of the catalyst of any one of embodiments 1 to 29.
38. The reactor tube of embodiment 37, wherein the second catalyst bed zone comprises, preferably consists of a second catalyst component according to any one of embodiments 1 and to 18 to 27.
39. The reactor tube of embodiment 37 or 38, wherein the volume of the first catalyst bed zone relative to the volume of the second catalyst bed zone is is in the range of from 0 to 100, preferably in the range of from 0.01 to 50, more preferably in the range of from 0.5 to 5.
40. The reactor tube of any one of embodiments 33 to 39, wherein the catalyst bed consists of the first catalyst bed zone and the second catalyst bed zone.
41. A reactor for converting a synthesis gas, comprising one or more reactor tubes according to any one of embodiments 30 to 40.
42. The reactor of embodiment 41, wherein the one or more tubes are vertically arranged.
43. The reactor of embodiment 42, wherein the one or more tubes have inlet means at the top allowing a gas stream to be passed into the reactor tube and outlet means at the bottom allowing a gas stream to be removed from the reactor tube.
44. The reactor of any one of embodiment 41 to 43, comprising two or more reactor tubes according to any one of embodiments 30 to 40, wherein the two or more reactor tubes are arranged in parallel.
45. The reactor of any one of embodiment 41 to 44, comprising temperature adjustment means allowing for isothermally converting the synthesis gas in the one or more reactor tubes.
46. Use of the catalyst according to any one of embodiments 1 to 29, optionally in combination with a second catalyst component according to any one of embodiments 1 and 18 to 27, for converting a synthesis gas comprising hydrogen and carbon monoxide, preferably for converting synthesis gas comprising hydrogen and carbon monoxide to one or more alcohols, preferably one or more of methanol and ethanol.
47. The use of embodiment 46, wherein for converting, the synthesis gas in passed into a reactor tube according to any one of embodiments 30 to 40, wherein said reactor tube is preferably comprised in a reactor according to any one of embodiments 41 to 45.
48. The use of embodiment 46 or 47, wherein the synthesis gas is passed into the reactor tube together with an inert gas, said inert gas preferably comprising argon.
49. A process for converting a synthesis gas comprising hydrogen and carbon monoxide to one or more of methanol and ethanol, said process comprising
   (i) providing a gas stream which comprises a synthesis gas stream comprising hydrogen and carbon monoxide;
   (ii) providing a catalyst according to any one of embodiments 1 to 29 and optionally a second catalyst component according to any one of embodiments 1 and 18 to 27;
   (iii) bringing the gas stream provided in (i) in contact with the catalyst provided in (ii) and optionally the second catalyst component according to any one of embodiments 1 and 18 to 27, obtaining a reaction mixture stream comprising one or more of methanol and ethanol.
50. The process of embodiment 49, wherein the catalyst provided in (ii) is comprised in a reactor tube according to any one of embodiments 30 to 40, wherein said reactor tube is preferably comprised in a reactor according to any one of embodiments 41 to 45, and wherein bringing the gas stream provided in (i) in contact with the catalyst provided in (ii) according to (iii) comprises passing the gas stream as feed stream into the reactor tube and through the catalyst bed comprised in the reactor tube, preferably from the top of the reactor tube to the bottom of the reactor tube, obtaining the reaction mixture stream comprising one or more of methanol and ethanol, said process further comprising removing the reaction mixture stream from the reactor tube.
51. The process of embodiment 49 or 50, wherein in the synthesis gas stream provided in (i), the molar ratio of hydrogen relative to carbon monoxide is in the range of from 0.5:1 to 10:1, preferably in the range of from 1:1 to 8:1, more preferably in the range of from 1.5:1 to 6:1, more preferably in the range of from 2:1 to 5:1.
52. The process of any one of embodiments 49 to 51, wherein in the synthesis gas stream provided in (i), the molar ratio of hydrogen relative to carbon monoxide is in the range of from 1:1 to 3:1, preferably in the range of from 1.5:1 to 2.5:1, more preferably in the range of from 1.75:1 to 2.25:1.
53. The process of any one of embodiments 49 to 51, wherein in the synthesis gas stream provided in (i), the molar ratio of hydrogen relative to carbon monoxide is in the range of from 4:1 to 6:1, preferably in the range of from 4.5:1 to 5.5:1, more preferably in the range of from 4.75:1 to 5.25:1.
54. The process of any one of embodiments 49 to 53, wherein at least 99 volume-%, preferably at least 99.5 volume-%, more preferably at least 99.9 volume-% of the synthesis gas stream according to (i) consist of hydrogen and carbon monoxide.
55. The process of any one of embodiments 49 to 54, wherein at least 80 volume-%, preferably at least 85 volume-%, more preferably at least 90 volume-%, more preferably from 90 to 99 volume-% of the gas stream provided in (i) consist of the synthesis gas stream.
56. The process of any one of embodiments 49 to 53, wherein the gas stream provided in (i) further comprises one or more inert gas preferably comprising, more preferably being argon.
57. The process of embodiment 56, wherein in the gas stream provided in (i), the volume ratio of the one or more inter gases relative to the synthesis gas stream is in the range of from 1:20 to 1:2, preferably in the range of from 1:15 to 1:5, more preferably in the range of from 1:12 to 1:8.
58. The process of embodiment 56 or 57, wherein at least 99 volume-%, preferably at least 99.5 volume-%, more preferably at least 99.9 volume-% of the gas stream provided in (i) consist of the synthesis gas stream and the one or more inert gases.
59. The process of any one of embodiments 49 to 58, wherein according to (iii), the gas stream is brought in contact with the catalyst at a temperature of the gas stream in the range of from 200 to 400° C., preferably in the range of from 220 to 350° C., more preferably in the range of from 240 to 310° C.
60. The process of any one of embodiments 49 to 59, wherein according to (iii), the gas stream is brought in contact with the catalyst at a pressure of the gas stream in the range of from 20 to 100 bar(abs), preferably in the range of from 40 to 80 bar(abs), more preferably in the range of from 50 to 60 bar(abs).
61. The process of any one of embodiments 49 to 60 insofar as being dependent on embodiment 50, wherein according to (iii), the gas stream is brought in contact with the catalyst at a gas hourly space velocity in the range of from 100 to 25,000 h⁻¹, preferably in the range of from 500 to 20,000 h⁻¹, more preferably in the range of from 1,000 to 10,000 h⁻¹, wherein the gas hourly space velocity is defined as the volume flow rate of the gas stream brought in contact with the catalyst divided by the volume of the catalyst bed.
62. The process of any one of embodiments 49 to 61, wherein prior to (iii), the catalyst provided in (i) is reduced.
63. The process of embodiment 62, wherein reducing the catalyst comprises bringing the catalyst in contact with a gas stream comprising hydrogen, wherein preferably at least 95 volume-%, preferably at least 98 volume-%, more preferably at least 99 weight-% of the gas stream consists of hydrogen.
64. The process of embodiment 63, wherein the gas stream comprising hydrogen is brought in contact with the catalyst at a temperature of the gas stream in the range of from 250 to 350° C., preferably in the range of from 275 to 325° C.
65. The process of embodiment 63 or 64, wherein the gas stream comprising hydrogen is brought in contact with the catalyst at a pressure of the gas stream in the range of from 10 to 100 bar(abs), preferably in the range of from 20 to 80 bar(abs).
66. The process of any one of embodiments 63 to 65 insofar as being dependent on embodiment 64, wherein the gas stream comprising hydrogen is brought in contact with the catalyst at a gas hourly space velocity in the range of from 500 to 15,000 h⁻¹, preferably in the range of from 1,000 to 10,000 h⁻¹, more preferably in the range of from 2,000 to 8,000 h⁻¹, wherein the gas hourly space velocity is defined as the volume flow rate of the gas stream brought in contact with the catalyst divided by the volume of the catalyst bed.
67. The process of any one of embodiments 63 to 68, wherein the catalyst is brought in contact with the gas stream comprising hydrogen fora period of time in the range of from 0.1 to 12 h, preferably in the range of from 0.5 to 6 h, more preferably in the range of from 1 to 3 h.
68. The process of any one of embodiments 49 to 67, wherein the selectivity of the conversion of the synthesis gas to one or more of methanol and ethanol exhibits a selectivity towards methane of at most 15% at a temperature during conversion of 260° C., wherein the selectivity is determined as described in Reference Example 2 herein.
69. The process of any one of embodiments 49 to 68, wherein the selectivity of the conversion of the synthesis gas to one or more of methanol and ethanol exhibits a selectivity towards methane of at most 25% at a temperature during conversion of 280° C., wherein the selectivity is determined as described in Reference Example 2 herein.
70. The process of any one of embodiments 49 to 69, wherein the selectivity of the conversion of the synthesis gas to one or more of methanol and ethanol exhibits a selectivity towards methane of at most 35% at a temperature during conversion of 300° C., wherein the selectivity is determined as described in Reference Example 2 herein.
71. The process of any one of embodiments 49 to 70, wherein the selectivity of the conversion of the synthesis gas to one or more of methanol and ethanol exhibits a selectivity towards acetic acid of less than 1% at a temperature during conversion of 260° C. or 280° C. or 300° C., wherein the selectivity is determined as described in Reference Example 2 herein.
72. The process of any one of embodiments 49 to 71, wherein the selectivity of the conversion of the synthesis gas to one or more of methanol and ethanol exhibits a selectivity towards the one or more of methanol and ethanol of at least 50% at a temperature during conversion of 260° C., wherein the selectivity is determined as described in Reference Example 2 herein.
73. The process of any one of embodiments 49 to 72, wherein the selectivity of the conversion of the synthesis gas to one or more of methanol and ethanol exhibits a selectivity towards the one or more of methanol and ethanol of at least 45% at a temperature during conversion of 280° C., wherein the selectivity is determined as described in Reference Example 2 herein.
74. A process for preparing the catalyst according to any one of embodiments 1 to 29, comprising
   (a) providing the first catalyst component according to any one of embodiments 1 to 17;
   (b) providing the second catalyst component according to any one of embodiments 1 and 18 to 27;
   (c) mixing the first catalyst component provided in (a) and the second catalyst component provided in (b).
75. The process of embodiment 74, wherein providing the first catalyst component according to (a) comprises preparing the first catalyst component by a method comprising
   (a.1) providing a source of the first porous oxidic substrate, preferably comprising subjecting the source of the first porous oxidic substrate to calcination;
   (a.2) providing a source of Rh, a source of Mn, a source of the alkali metal, preferably Li, and a source of Fe;
   (a.3) impregnating the preferably calcined source of the first porous oxidic substrate obtained from (a.1) with the sources provided in (a.2);
   (a.4) calcining the impregnated source of the first porous oxidic substrate, preferably after drying.
76. The process of embodiment 75, wherein according to (a.1), the first porous oxidic substrate is calcined, preferably in a gas atmosphere at a temperature of the gas atmosphere in the range of from 450 to 650° C., preferably in the range of from 500 to 600° C., wherein the gas atmosphere preferably comprises oxygen, more preferably is oxygen, air, or lean air.
77. The process of embodiment 75 or 76, wherein according to (a.1), the source of the first porous oxidic substrate comprises silica, zirconia, titania, alumina, a mixture of two or more of silica, zirconia, titania, and alumina, or a mixed oxide of two or more of silicon, zirconium, titanium, and aluminum, wherein more preferably, the first porous oxidic substrate comprises silica.
78. The process of embodiment 77, wherein the silica has a BET specific surface area in the range of 500 to 550 m²/g determined as described in Reference Example 1.1 herein; a total intrusion volume in the range of from 0.70 to 0.80 mL/g, determined as described in Reference Example 1.2 herein; an average pore diameter in the range of from 55 to 65 Angstrom, determined as described in Reference Example 1.3 herein.
79. The process of any one of embodiment 75 to 78,
   wherein the source of Rh comprises a Rh salt, preferably an inorganic Rh salt, more preferably a Rh nitrate, wherein more preferably, the source of Rh is a Rh nitrate;
   wherein the source of Mn comprises a Mn salt, preferably an inorganic Mn salt, more preferably a Mn nitrate, wherein more preferably, the source of Mn is a Rh nitrate;
   wherein the source of the alkali metal, preferably Li, comprises an alkali metal salt, preferably a Li salt, preferably an inorganic alkali metal salt, preferably an inorganic Li salt, more preferably an alkali metal nitrate, preferably a Li nitrate, wherein more preferably, the source of the alkali metal is an alkali metal nitrate, more preferably a Li nitrate;
   wherein the source of Fe comprises a Fe salt, preferably an inorganic Fe salt, more preferably a Fe nitrate, wherein more preferably, the source of Fe is a Fe nitrate.
80. The process of any one of embodiments 75 to 79, wherein (a.2) comprises preparing an aqueous solution comprising the source of Rh, the source of Mn, the source of the alkali metal, preferably Li, and the source of Fe, and wherein (a.3) comprises impregnating the source of the first porous oxidic substrate obtained from (a.1) with said aqueous solution.
81. The process of any one of embodiments 75 to 80, wherein in (a.4), the impregnated source of the first porous oxidic substrate obtained from (a.3) is calcined in a gas atmosphere at a temperature of the gas atmosphere in the range of from 180 to 250° C., preferably in the range of from 190 to 220° C., wherein the gas atmosphere preferably comprises oxygen, more preferably is oxygen, air, or lean air, preferably after drying in a gas atmosphere at a temperature of the gas atmosphere in the range of from 90 to 150° C., preferably in the range of from 100 to 130° C., wherein the gas atmosphere preferably comprises oxygen, more preferably is oxygen, air, or lean air.
82. The process of any one of embodiments 74 to 81, wherein providing the second catalyst component according to (b) comprises preparing the second catalyst component by a method comprising
   (b.1) providing a source of the second porous oxidic substrate, preferably comprising subjecting the source of the second porous oxidic substrate to calcination;
   (b.2) providing a source of Cu, a source of the transition metal other than Cu, preferably Zn;
   (b.3) impregnating the preferably calcined source of the second porous oxidic substrate obtained from (a.1) with the sources provided in (a.2);
   (b.4) calcining the impregnated source of the second porous oxidic substrate, preferably after drying.
83. The process of embodiment 82, wherein according to (b.1), the second porous oxidic substrate is calcined, preferably in a gas atmosphere at a temperature of the gas atmosphere in the range of from 750 to 950° C., preferably in the range of from 800 to 900° C., wherein the gas atmosphere preferably comprises oxygen, more preferably is oxygen, air, or lean air.
84. The process of embodiment 82 or 83, wherein according to (b.1), the source of the first porous oxidic substrate comprises silica, zirconia, titania, alumina, a mixture of two or more of silica, zirconia, titania, and alumina, or a mixed oxide of two or more of silicon, zirconium, titanium, and aluminum, wherein more preferably, the first porous oxidic substrate comprises silica.
85. The process of embodiment 84, wherein the silica has a BET specific surface area in the range of 500 to 550 m²/g determined as described in Reference Example 1.1 herein; a total intrusion volume in the range of from 0.70 to 0.80 mL/g, determined as described in Reference Example 1.2 herein; an average pore diameter in the range of from 55 to 65 Angstrom, determined as described in Reference Example 1.3 herein.
86. The process of any one of embodiments 82 to 85,
   wherein the source of Cu comprises a Cu salt, preferably an inorganic Cu salt, more preferably a Cu nitrate, wherein more preferably, the source of Cu is a Cu nitrate;
   wherein the source of the transition metal other than Cu, preferably Zn, comprises a salt of the transition metal other than Cu, preferably a Zn salt, preferably an inorganic salt of the transition metal other than Cu, preferably an inorganic Zn salt, more preferably a nitrate of the transition metal other than Cu, preferably a Zn nitrate, wherein more preferably, the source of the transition metal other than Cu is a nitrate of the transition metal other than Cu, more preferably a Zn nitrate.
87. The process of any one of embodiments 82 to 86, wherein (b.2) comprises preparing an aqueous solution comprising the source of Cu and the source of the transition metal other than Cu, preferably Zn, and wherein (b.3) comprises impregnating the source of the second porous oxidic substrate obtained from (b.1) with said aqueous solution.
88. The process of any one of embodiments 82 to 87, wherein in (b.4), the impregnated source of the second porous oxidic substrate obtained from (b.3) is calcined in a gas atmosphere at a temperature of the gas atmosphere in the range of from 300 to 500° C., preferably in the range of from 350 to 450° C., wherein the gas atmosphere preferably comprises oxygen, more preferably is oxygen, air, or lean air, preferably after drying in a gas atmosphere at a temperature of the gas atmosphere in the range of from 80 to 140° C., preferably in the range of from 90 to 120° C., wherein the gas atmosphere preferably comprises oxygen, more preferably is oxygen, air, or lean air.
89. A first catalyst component, preferably the first catalyst component according to any one of embodiments 1 to 17, obtainable or obtained or preparable or prepared by a process according to any one of embodiments 75 to 81.
90. A second catalyst component, preferably the second catalyst component according to any one of embodiments 1 and 18 to 27, obtainable or obtained or preparable or prepared by a process according to any one of embodiments 82 to 88.
91. A porous oxidic substrate, comprising supported thereon Rh, Mn, Li and Fe, having a chlorine content in the range of from 0 to 100 weight-ppm, based on the total weight of said substrate, Rh, Mn, Li and Fe.
92. The porous oxidic substrate of embodiment 91, being silica comprising supported thereon Rh, Mn, Li and Fe.
93. The porous oxidic substrate of embodiment 91 or 92,
   having a Rh content, calculated as elemental Rh, in the range of from 2.0 to 3.0 weight-%, preferably in the range of from 2.1 to 2.8 weight-%, more preferably in the range of from 2.2 to 2.6 weight-%;
   having a Mn content, calculated as elemental Mn, in the range of from 0.40 to 0.70 weight-%, preferably in the range of from 0.45 to 0.60 weight-%, more preferably in the range of from 0.50 to 0.55 weight-%;
   having a Fe content, calculated as elemental Li, in the range of from 0.35 to 0.65 weight-%, preferably in the range of from 0.40 to 0.55 weight-%, more preferably in the range of from 0.45 to 0.50 weight-%;
   having a Li content, calculated as elemental Fe, in the range of from 0.10 to 0.40 weight-%, preferably in the range of from 0.15 to 0.30 weight-%, more preferably in the range of from 0.20 to 0.25 weight-%;
   based on the total weight of the porous oxidic substrate, comprising supported thereon Rh, Mn, Li and Fe.
94. The porous oxidic substrate of any one of embodiments 91 to 93, wherein at least 99 weight-%, preferably at least 99.9 weight-%, more preferably at least 99.99 weight-% of the porous oxidic substrate consist of the porous oxidic substrate, Rh, Mn, Li and Fe.
95. The porous oxidic substrate of any one of embodiments 91 to 94, having a BET specific surface area in the range of from 350 to 450 m²/g, preferably in the range of from 375 to 425 m²/g, determined as described in Reference Example 1.1 herein.
96. The porous oxidic substrate of any one of embodiments 91 to 95, obtainable or obtained or preparable or prepared by a process according to any one of embodiments 75 to 80.
97. A process for reducing the catalyst of any one of embodiments 1 to 29, comprising bringing the catalyst in contact with a gas stream comprising hydrogen, wherein preferably at least 95 volume-%, preferably at least 98 volume-%, more preferably at least 99 weight-% of the gas stream consists of hydrogen.
98. The process of embodiment 97, wherein the gas stream comprising hydrogen is brought in contact with the catalyst at a temperature of the gas stream in the range of from 250 to 350° C., preferably in the range of from 275 to 325° C., preferably at a pressure of the gas stream in the range of from 10 to 100 bar(abs), more preferably in the range of from 20 to 80 bar(abs).
99. The process of embodiment 97 to 98, wherein the catalyst is brought in contact with the gas stream comprising hydrogen for a period of time in the range of from 0.1 to 12 h, preferably in the range of from 0.5 to 6 h, more preferably in the range of from 1 to 3 h.
100. A catalyst, obtainable or obtained or preparable or prepared by a process according to any one of embodiments 97 to 99.

In the context of the present invention, a ratios such as a weight ratio or a volume ratio of a first component or compound X relative to a second component or compound X which is described as being in a range of from x to y is to be understood as being in the range of from x:1 to y:1.

The invention is further illustrated by the following Reference Examples, Examples and Comparative Examples.

### EXAMPLES

### Reference Example 1: Determination of Characteristics of Materials

### Reference Example 1.1: Determination of the BET Specific Surface Area

The BET specific surface area was determined via nitrogen physisorption at 77 K according to the method disclosed in DIN 66131.

### Reference Example 1.2: Determination of the Total Intrusion Volume

The total intrusion volume was determined by Hg-porosimetry at 59.9 psi (pounds per square inch) according to DIN 66133. It is 1.6825 mL/g for the first catalyst component according to Example 1.1 and 1.0150 mL/g for the second catalyst component according to Example 1.2.

### Reference Example 1.3: Determination of the Average Pore Diameter

The average pore diameter was determined by Hg-porosimetry according to DIN 66133. It is 0.01881 micrometer the first catalyst component according to Example 1.1 and 0.02109 micrometer for the second catalyst component according to Example 1.2.

### Reference Example 2: Determination of Selectivities and Yields

The selectivity with respect to a given compound A, S(A), was determined via GC chromatography analysis.

In particular, the selectivity S(A) was calculated according to following formula:

S(A)/%=[Y(A)/X(CO)]*100

Y(A) is the yield with respect to the compound A and X is the conversion of carbon monoxide.

Conversion X(CO)

The conversion X(CO) in % is defined as

X(CO)/%=[(Rₘₒₗ(CO in)−Rₘₒₗ(CO))/Rₘₒₗ(CO in)]*100

For a given reaction tube, the (inlet) molar flow rate Rₘₒₗ(CO in) is defined as

Rₘₒₗ(CO in)/(mol/h)=F(CO)/V

wherein

F(CO)/(I/h) is the flow rate of carbon monoxide into the reaction tube;

V/(I/mol) is the mole volume.

Further, the (outlet) molar flow rate Rₘₒₗ(CO) is defined as

Rₘₒₗ(CO)/(mol/h)=R_{C}(CO)/(M(C)*N_{C}(CO))

wherein the carbon flow rate R_{C}(CO) in (g(C)/h) is defined as

R_{C}(CO)/(g(C)/h)=(F(CO)/R(CO))*F

wherein

F(CO) is the peak area of the compound CO measured via gas chromatography,

R(CO) is the response factor obtained from gas chromatography calibration,

F is the measured flow rate of the gas phase; and

wherein

M(C) is the molecular weight of C;

N_{C}(CO) is the number of carbon atoms of CO, i.e. N_{C}(CO)=1.

Yield Y(A)

The yield Y(A) in % is defined as

Y(A)/%=(R_{C}(A)/R_{C}(CO in))*100

The (outlet) carbon flow rate R_{C}(A) in g(C)/h is defined as

R_{C}(A)/(g(C)/h)=(F(A)/R(A))*F

wherein

F(A) is the peak area of the compound A measured via gas chromatography,

R(A) is the response factor obtained from gas chromatography calibration,

F is the measured flow rate of the gas phase.

The (inlet) flow rate R_{C}(CO in) in g(C)/h is defined as

R_{C}(CO in)/g(C)/h=Rₘₒₗ(CO in)*M(C)*N_{C}(CO)

wherein

Rₘₒₗ(CO in) is as defined above,

M(C) is as defined above;

N_{C}(CO) is the number of carbon atoms of compound CO, i.e. N_{C}(CO)=1.

### Example 1: Preparation of the Catalyst of the Invention

### Example 1.1: Preparation of the First Catalytic Component

A colloidal silica gel (Davisil® 636 from Sigma-Aldrich, powder, having a particle size in the range of from 250 to 300 micrometer, a purity of at least 99%, an average pore diameter of 60 Angstrom, a total intrusion volume of 0.75 mL/g, and BET specific surface area of 515 m²/g) was calcined for 6 hours at 550° C. in a muffle furnace to obtain a BET surface area of 546 m²/g. An aqueous solution containing 5.79 g rhodium nitrate solution (10.09 weight-% Rh), 0.58 g manganese nitrate tetrahydrate (Mn(NO₃)₂ 4H₂O), 0.76 g iron nitrate nonahydrate (Fe(NO₃)₃ 9H₂O) and 0.60 g lithium nitrate was added dropwise to 20 g of the calcined silica gel. The impregnated support was then dried at 120° C. for 3 hours (heating rate: 3 K/min) and calcined in air at 200° C. for 3 hours in a muffle furnace (heating rate: 2 K/min).

### Example 1.2: Preparation of the Second Catalytic Component

A colloidal silica gel (Davisil® 636 from Sigma-Aldrich) was calcined for 12 hours at 850° C. in a muffle furnace to obtain a BET specific surface area of 320 m²/g. An aqueous solution containing 3.75 g copper nitrate trihydrate (Cu(NO₃)₂ 3H₂O) and 4.59 g zinc nitrate hexahydrate (Zn(NO₃)₂ 6H₂O) was added dropwise to 20 g of the calcined Davisil®. The impregnated support was then dried at 110° C. for 3 hours (heating rate: 3 K/min) and calcined in air at 400° C. for 3 hours in a muffle furnace (heating rate: 2 K/min).

### Comparative Example 1: Preparation of a Catalyst Having a Non-Inventive First Catalytic Component

A first catalyst component was prepared as follows: A colloidal silica gel (Davisil® 636 from Sigma-Aldrich) was calcined for 6 hours at 550° C. in a muffle furnace to obtain a BET specific surface area of 546 m²/g. An aqueous solution containing 11.66 g rhodium nitrate solution (10.09 weight-% Rh), 2.94 g manganese nitrate tetrahydrate (Mn(NO₃)₂×4H₂O) and 1.52 g iron nitrate nonahydrate (Fe(NO₃)₃×9 H₂O) was added dropwise to 40 g of the calcined Davisil®. The impregnated support was then dried at 120° C. for 3 hours (heating rate: 3 K/min) and calcined in air at 350° C. for 3 hours in a muffle furnace (heating rate: 2 K/min).

### Comparative Example 2: Preparation of a Catalyst Having a Non-Inventive First Catalytic Component

According to the teaching of US 2015/0284306 A1, a first catalyst component was prepared as follows: A colloidal silica gel (Davisil® 636 from Sigma-Aldrich) was calcined for 12 hours at 725° C. in a muffle furnace to obtain a BET specific surface area of 451 m²/g. An aqueous solution containing 0.49 g of titanium(IV)bis(ammoniumlactato)dihydroxide solution (50 weight-% from Sigma-Aldrich) was added dropwise to 20 g of the calcined Davisil®. The impregnated support was then dried at 110° C. for 3 hours (heating rate: 3 K/min) and calcined at 450° C. for 3 hours in a muffle furnace (heating rate: 2 K/min). Subsequently, this intermediate was impregnated dropwise with a second aqueous solution, which contained 1.78 g rhodium chloride trihydrate (RhCl₃ 3H₂O), 0.88 g manganese chloride tetrahydrate (MnCl₂ 4H₂O) and 0.06 g lithium chloride (LiCl). The volume of both aqueous solutions equated to 100% water uptake. The impregnated support was then dried at 110° C. for 3 hours (heating rate: 3 K/min) and calcined under air at 450° C. for 3 hours in a rotary calciner (heating rate: 1 K/min).

The individual materials had the compositions as shown in Table 1 below.

TABLE 1
Compositions of the prepared materials

| | Catalyst component | | | | | | | | |
|---|---|---|---|---|---|---|---|---|---|
| | Rh/ | Mn/ | Fe/ | Li/ | Ti/ | Cl/ | Cu/ | Zn/ | BET/ |
| | wt-% | wt-% | wt-% | wt-% | wt-% | wt-% | wt-% | wt-% | m²/g |
| Comparative | 2.5 | 1.1 | 0 | 0.04 | 0.18 | 2.7 | 0 | 0 | 397 |
| Example 1 | | | | | | | | | |
| Example 1.1 | 2.4 | 0.53 | 0.49 | 0.25 | 0 | 0 | 0 | 0 | 397 |
| Comparative | 2.5 | 1.1 | 0 | 0.04 | 0.18 | 2.7 | 0 | 0 | 397 |
| Example 2 | | | | | | | | | |
| Example 1.2 | 0 | 0 | 0 | 0 | 0 | 0 | 3.8 | 4.1 | 247 |

### Example 3: Catalytic Testing

### Example 3.1: Catalyst Reaction in Single-Catalyst Bed Reactor

The reactions were performed in continuous flow a stainless steel reactor in the gas phase. The catalyst bed was not diluted with inert material. Particle fractions were used with a dimension of 250-315 micrometer. The catalyst particles were placed into the isothermal zone of the reactors. The non-isothermal zone of the reactor was filled with inert corundum (alpha-Al₂O₃). Three reaction temperatures were adjusted during the continuous experiment (260° C., 280° C., and 300° C.). The H₂/CO ratio of the synthesis gas was varied between 5 and 2 for each reaction temperature, giving 6 parameter variations in total. The reaction pressure was kept constant at 54 bar(abs) for each experiment. The total mass (g) for each catalyst placed into the reactor was:
0.636 g of the first catalyst component of Comparative Example 2 (RhMnLiTiCl/SiO₂)
0.578 g of the first catalyst component of Comparative Example 1 (RhMnFeCl/SiO₂)
0.602 g of the first catalyst component of Example 1.1 (RhMnFeLi/SiO₂)

Each catalyst was subjected to an in-situ reduction in H₂ for 2 h at 310° C. prior to the reaction. Synthesis gas with CO and H₂ contained 10 volume-% Ar as the internal standard for online gas chromatography (GC) analysis. Reaction was carried out with a gaseous hourly space velocity of 3750 h⁻¹. Data were collected for at least 5 hours on stream. A summary of the reaction conditions and catalytic performance of the individual catalyst is given in Table 2. Selectivities are reported in carbon atom %, determined as described in Reference Example 2.

TABLE 2
Catalytic reaction in single-catalyst bed reactor

| | Catalyst | | | | | | | | |
|---|---|---|---|---|---|---|---|---|---|
| | T/ | H₂/ | X(CO)/ | S_CO₂/ | S_MeOH/ | S_EtOH/ | S_CH₄/ | S_AA/ | S_HAc/ |
| | ° C. | CO ^{a)} | % ^{b)} | % ^{c)} | % ^{d)} | % ^{e)} | % ^{f)} | % ^{g)} | % ^{h)} |
| Comp. | 260 | 5 | 28 | 3 | 6 | 30 | 53 | 0 | 1 |
| Ex. 1 | 260 | 2 | 10 | 2 | 4 | 33 | 44 | 0 | 2 |
| | 280 | 5 | 44 | 6 | 12 | 22 | 56 | 0 | 0 |
| | 280 | 2 | 18 | 5 | 7 | 29 | 47 | 1 | 1 |
| | 300 | 5 | 72 | 7 | 10 | 14 | 65 | 0 | 0 |
| | 300 | 2 | 31 | 7 | 8 | 24 | 53 | 1 | 1 |
| Ex. 1.1 | 260 | 5 | 14 | 24 | 15 | 31 | 21 | 0 | 0 |
| | 260 | 2 | 5 | 20 | 6 | 31 | 22 | 0 | 3 |
| | 280 | 5 | 35 | 28 | 9 | 26 | 28 | 1 | 0 |
| | 280 | 2 | 13 | 24 | 5 | 25 | 24 | 2 | 3 |
| | 300 | 5 | 75 | 29 | 5 | 21 | 37 | 2 | 0 |
| | 300 | 2 | 28 | 30 | 3 | 20 | 30 | 2 | 1 |
| Comp. | 260 | 5 | 62 | 0 | 0 | 19 | 37 | 15 | 0 |
| Ex. 2 | 260 | 2 | 19 | 0 | 0 | 8 | 25 | 25 | 0 |
| | 280 | 5 | 91 | 1 | 1 | 24 | 54 | 3 | 0 |
| | 280 | 2 | 35 | 1 | 0 | 11 | 33 | 20 | 0 |
| | 300 | 5 | 89 | 3 | 1 | 24 | 61 | 1 | 1 |
| | 300 | 2 | 41 | 3 | 1 | 17 | 40 | 15 | 0 |

^{a)} molar ratio of hydrogen relative to oxygen in the synthesis gas stream
^{b)} conversion of carbon monoxide
^{c)} selectivity towards carbon dioxide
^{d)} selectivity towards methanol
^{e)} selectivity towards ethanol
^{f)} selectivity towards methane
^{g)} selectivity towards acetaldehyde
^{h)} selectivity towards acetic acid

### Results of Example 3.1

As shown above, in Table 2, the inventive first catalyst component according to Example 1.1 exhibits a much better (much lower) selectivity with regard to the by-product acetaldehyde than the catalyst according to comparative example 2. In particular, for each temperature and for each ratio H₂/CO in the feed stream, the inventive first catalyst component according to Example 1.1 exhibits a much better (much lower) selectivity with regard to the by-product methane than both the catalyst according to comparative example 1 and the catalyst according to compartitive example 2.

### Example 3.2: Catalyst Reaction in Two-Catalyst Bed Reactor

The reactions were performed in the gas phase using 16-fold unit with stainless steel reactors. The catalyst bed was not diluted with inert material. Particle fractions were used with a dimension of 250-315 micrometer. The catalyst particles were placed into the isothermal zone of the reactors. The non-isothermal zone of the reactor was filled with inert corundum (alpha-Al₂O₃). The catalyst bed was designed so that a physical mixture of two catalysts is used: The synthesis gas meets at the entrance of the reactor initially a physical mixture of two catalyst particles, the first and the second catalyst components (CuZn/SiO₂ catalyst component+Rh-based catalyst component), and then the partially converted gas meets catalyst particles which consist only of the second catalyst component (CuZn/SiO₂ particles). Three reaction temperatures were varied during the continuous experiment (260° C., 280° C., and 300° C.). The H₂/CO ratio of the synthesis gas was varied between 5 and 2 between each reaction temperature, giving 6 variations in total. The reaction pressure was kept constant at 54 bar(abs). The total mass (g) for each catalyst for the top two-catalyst bed was as following:
top mixture:
   0.348 g of the first component of Comparative Example 1 (RhMnLiTiCl/SiO₂)
   0.104 g of the second component of Example 1.2 (CuZn/SiO₂)
bottom mixture:
   0.255 g of the second component of Example 1.2 (CuZn/SiO₂)
top mixture:
   0.317 g of the first component of Comparative Example 2 (RhMnFeCl/SiO₂)
   0.105 g of the second component of Example 1.2 (CuZn/SiO₂)
bottom mixture:
   0.253 g of the second component of Example 1.2 (CuZn/SiO₂)
top mixture:
   0.334 g of the first component of Example 1.1 (RhMnFeLi/SiO₂)
   0.106 g of the second component of Example 1.2 (CuZn/SiO₂)
bottom mixture
   0.256 g of the second component of Example 1.2 (CuZn/SiO₂).

Each catalyst mixture was subjected to in-situ reduction in H₂ for 2 h at 310° C. prior to reaction. Synthesis gas with CO and H₂ contained 10 volume-% Ar as the internal standard for online gas chromatography (GC) analysis. Reaction was carried out under a gaseous hourly space velocity of 3750 h⁻¹. Data were collected for at least 5 hours on stream. The reaction conditions and catalytic performance for each catalytic mixture are given in Table 3. Selectivities are reported in carbon atom %, determined as described in Reference Example 2.

TABLE 3
Catalytic reaction in two-catalyst bed reactor

| | Catalyst | | | | | | | | |
|---|---|---|---|---|---|---|---|---|---|
| | T/ | H₂/ | X(CO)/ | S_CO₂/ | S_MeOH/ | S_EtOH/ | S_CH₄/ | S_AA/ | S_HAc/ |
| | ° C. | CO ^{a)} | % ^{b)} | % ^{c)} | % ^{d)} | % ^{e)} | % ^{f)} | % ^{g)} | % ^{h)} |
| Comp. | 260 | 5 | 20 | 12 | 12 | 31 | 42 | 0 | 0 |
| Ex. 1 | 260 | 2 | 7 | 13 | 8 | 38 | 34 | 0 | 0 |
| and | 280 | 5 | 29 | 9 | 16 | 23 | 49 | 0 | 0 |
| Ex. 1.2 | 280 | 2 | 11 | 9 | 12 | 33 | 41 | 0 | 0 |
| | 300 | 5 | 47 | 7 | 15 | 17 | 58 | 0 | 0 |
| | 300 | 2 | 19 | 9 | 12 | 26 | 48 | 1 | 0 |
| Ex. 1.1 | 260 | 5 | 10 | 23 | 30 | 32 | 13 | 0 | 0 |
| and | 260 | 2 | 4 | 28 | 21 | 33 | 13 | 0 | 0 |
| Ex. 1.2 | 280 | 5 | 20 | 26 | 19 | 30 | 22 | 0 | 0 |
| | 280 | 2 | 8 | 29 | 12 | 34 | 19 | 0 | 0 |
| | 300 | 5 | 40 | 27 | 10 | 26 | 32 | 0 | 0 |
| | 300 | 2 | 17 | 28 | 7 | 31 | 26 | 1 | 0 |
| Comp. | 260 | 5 | 13 | 0 | 0 | 39 | 31 | 0 | 3 |
| Ex. 2 | 260 | 2 | 4 | 0 | 0 | 36 | 23 | 0 | 5 |
| and | 280 | 5 | 23 | 2 | 1 | 42 | 39 | 0 | 1 |
| Ex. 1.2 | 280 | 2 | 9 | 3 | 1 | 42 | 27 | 0 | 2 |
| | 300 | 5 | 38 | 3 | 2 | 36 | 50 | 0 | 0 |
| | 300 | 2 | 17 | 4 | 2 | 41 | 36 | 1 | 1 |

^{a)} molar ratio of hydrogen relative to oxygen in the synthesis gas stream
^{b)} conversion of carbon monoxide
^{c)} selectivity towards carbon dioxide
^{d)} selectivity towards methanol
^{e)} selectivity towards ethanol
^{f)} selectivity towards methane
^{g)} selectivity towards acetaldehyde
^{h)} selectivity towards acetic acid

### Results of Example 3.2

As shown above, in Table 2, the catalyst comprising the inventive first and second catalyst components exhibits a much better (i.e. much lower) selectivity with regard to the by-product acetic acid than the catalyst according the comparative first compound of Example 2. In particular, for each temperature and for each ratio H₂/CO in the feed stream, the catalyst comprising the inventive first and second catalyst components exhibits a much better (much lower) selectivity with regard to the by-product methane than the catalyst comprising the comparative first catalyst component of Comparative Example 1 as well as the catalyst comprising the comparative first catalyst component of Comparative Example 2.

### CITED PRIOR ART

US 2015/0284306 A1

1.-20. (canceled)

21. A catalyst for converting a synthesis gas, said catalyst comprising a first catalyst component and a second catalyst component, wherein the first catalyst component comprises, supported on a first porous oxidic substrate, Rh, Mn, an alkali metal M and Fe, and wherein the second catalyst component comprises, supported on a second porous oxidic support material, Cu and a transition metal other than Cu.

22. The catalyst of claim 21, wherein in the first catalyst component,
the molar ratio of Rh, calculated as elemental Rh, relative to Mn, calculated as elemental Mn, is in the range of from 0.1 to 10;
the molar ratio of Rh, calculated as elemental Rh, relative to Fe, calculated as elemental Fe, is in the range of from 0.1 to 10; and
the molar ratio of Rh calculated as elemental Rh, relative to the alkali metal M, calculated as elemental M, is in the range of from 0.1 to 5.

23. The catalyst of claim 21, wherein the alkali metal M comprised in the first catalyst component is one or more of Na, Li, K, Rb, Cs.

24. The catalyst of claim 21, wherein at least 99 weight-% of the first catalyst component consist of Rh, Mn, the alkali metal M, Fe, O, and the first porous oxidic substrate.

25. The catalyst of claim 21, wherein the first porous oxidic substrate comprises silica, zirconia, titania, alumina, a mixture of two or more of silica, zirconia, titania, and alumina, or a mixed oxide of two or more of silicon, zirconium, titanium, and aluminum, wherein in the first catalyst component, the weight ratio of Rh, calculated as elemental Rh, relative to the first porous oxidic substrate is in the range of from 0.001:1 to 4.000:1.

26. The catalyst of claim 21, wherein the first catalyst component has a BET specific surface area in the range of from 250 to 500 m²/g, a total intrusion volume in the range of from 0.1 to 5 mL/g, and an average pore diameter in the range of from 0.001 to 0.5 micrometer.

27. The catalyst of claim 21, wherein in the second catalyst component, the transition metal other than Cu is one or more of Cr and Zn, wherein the molar ratio of Cu, calculated as elemental Cu, relative to the transition metal other than Cu, calculated as elemental metal, is in the range of from 0.1 to 5.

28. The catalyst of claim 21, wherein at least 99 weight-% of the second catalyst component consist of Cu, the transition metal other than Cu, O, and the second porous oxidic substrate.

29. The catalyst of claim 21, wherein the second porous oxidic substrate comprises silica, zirconia, titania, alumina, a mixture of two or more of silica, zirconia, titania, and alumina, or a mixed oxide of two or more of silicon, zirconium, titanium, and aluminum, wherein the weight ratio of Cu, calculated as elemental Cu, relative to the second porous oxidic substrate is in the range of from 0.001 to 0.5.

30. The catalyst of claim 21, wherein the second catalyst component has a BET specific surface area in the range of from 100 to 500 m²/g, a total intrusion volume in the range of from 0.1 to 10 mL/g, and an average pore diameter in the range of from 0.001 to 5 micrometer.

31. The catalyst of claim 21, wherein the weight ratio of the first catalyst component relative to the second catalyst component is in the range of from 1 to 10.

32. The catalyst of claim 21, wherein at least 99 weight-% of the catalyst consist of the first catalyst component and the second catalyst component.

33. A reactor tube for converting a synthesis gas, comprising a catalyst bed which comprises the catalyst of claim 21.

34. The rector tube of claim 33, being vertically arranged, comprising two or more catalyst bed zones, wherein a first catalyst bed zone is arranged on top of a second catalyst bed zone, wherein the first catalyst bed zone comprises the catalyst, and wherein the second catalyst bed zone comprises the second catalyst component.

35. The reactor tube of claim 34, wherein the volume of the first catalyst bed zone relative to the volume of the second catalyst bed zone is in the range of from 0 to 100.

36. A method for converting a synthesis gas comprising hydrogen and carbon monoxide to one or more alcohols, comprising utilizing the catalyst according to claim 21.

37. A process for converting a synthesis gas comprising hydrogen and carbon monoxide to one or more of methanol and ethanol, said process comprising
(i) providing a gas stream which comprises a synthesis gas stream comprising hydrogen and carbon monoxide;
(ii) providing the catalyst according to claim 21;
(iii) bringing the gas stream provided in (i) in contact with the catalyst provided in (ii), obtaining a reaction mixture stream comprising one or more of methanol and ethanol.

38. The process of claim 37, wherein prior to (iii), the catalyst provided in (i) is reduced, wherein reducing the catalyst comprises bringing the catalyst in contact with a gas stream comprising hydrogen.

39. A process for preparing the catalyst according to claim 21, comprising
(a) providing the first catalyst component;
(b) providing the second catalyst component;
(c) mixing the first catalyst component provided in (a) and the second catalyst component provided in (b).

40. The process of claim 39, wherein providing the first catalyst component according to (a) comprises preparing the first catalyst component by a method comprising
(a.1) providing a source of the first porous oxidic substrate;
(a.2) providing a source of Rh, a source of Mn, a source of the alkali metal, and a source of Fe;
(a.3) impregnating the source of the first porous oxidic substrate obtained from (a.1) with the sources provided in (a.2);
(a.4) calcining the impregnated source of the first porous oxidic substrate,
and wherein providing the second catalyst component according to (b) comprises preparing the second catalyst component by a method comprising
(b.1) providing a source of the second porous oxidic substrate;
(b.2) providing a source of Cu, a source of the transition metal other than Cu;
(b.3) impregnating the source of the second porous oxidic substrate obtained from (a.1) with the sources provided in (a.2);
(b.4) calcining the impregnated source of the second porous oxidic substrate.
